(19) United States
(12) Patent Application Publication
(10) Pub. No.: US 2007/0043407 A1
(43) Pub. Date: Feb. 22, 2007

(54) Hand-held photon shining apparatus

(76) Inventor: Wun-Chen Lin, Walnut, CA (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/471,522

(22) Filed: Jun. 21, 2006

(30) Foreign Application Priority Data
Aug. 19, 2005 (TW) 94214271

(51) Int. Cl. A61N 5/06

(52) U.S. Cl. 607/88

(57) ABSTRACT

A hand-held photon shining apparatus can be held by hand to cover the photon shining apparatus on the human body to perform a photon shining operation. The hand-held photon shining apparatus includes a main body, a battery receiving space, a switch, a concave slot, and at least one light-emitting element. A bottom concave part is formed on the bottom surface. The battery receiving space is used for receiving at least one battery. The switch is located at the front half part of the top surface of the main body. The concave slot is located within the bottom concave part. The light-emitting element is located in the slot bottom of the concave slot. Via holding the main body by hand and covering the slot opening of the concave slot on the treatment area of human body, the light-emitting element is controlled to shine the treatment area, when the switch is pressed.

### BACKGROUND OF THE INVENTION

1. Field of the Invention

The present invention relates to a hand-held photon shining apparatus. In particular, this invention relates to a photon shining apparatus that is handheld by the user to cover the photon shining apparatus on a patient's to perform a photon shining operation for cosmetic and/or medical operations.

2. Description of the Related Art

The photomodulation is commonly applied for dermatologic medical treatments, such as wrinkle reduction, acne reduction, hair growth stimulation, hair removal etc. However, the dermatologic medical instruments are usually large in size.

U.S. Pat. No. 6,063,108 discloses a “low energy photon therapy apparatus and a method thereof”. It uses a matrix light source 10, as shown in FIG. 1, to shine photons on an injured animal's tissue to treat the damaged tissue. The apparatus includes a power source, a central processor having a variety of photo parameters and a medical protocol, and a detector. It provides a plurality of photo parameters to control the matrix light source 10 to generate a light having a specified wavelength range and medical effect. The wavelength range of the light having the medical effect that is disclosed by this patent is from 360 n to 440 nm, from 630 nm to 700 nm, from 740 nm to 760 nm, and from 800 nm to 1100 nm.

U.S. Pat. No. 6,645,230 discloses a light source having a medical effect and its embodiments. The wavelength range of the light having the medical effect is from 550 nm to 660 nm. The embodiments are shown in the FIG. 2A and FIG. 2B. The light source 20 of the photon treating apparatus can cover part or all of the human body 22.

Although some hand-held photon shining apparatuses have been disclosed (not shown in the figure), all of the apparatuses are not ergonomically designed. It is difficult for a user to hold the devices with their hand. After the user has been operating the apparatus for a long time, the user's hand becomes tired and/or suffers muscular pains. Moreover, when the user operates the apparatus, there is a distance between the apparatus and the part of the skin where the device is shining at. Because there is not any steadying base, the user cannot ensure that the apparatus is shining on the exact desired position or not. The shining location can be easily moved. After the apparatus has been shifted, the user does not know the situation and the medical effect is not assured.

U.S. Pat. No. 6,497,719 discloses “an apparatus for exciting the hair to grow and a method thereof”. It uses an emitter having a comb-shape to push aside hairs to allow a laser light to shine on the scalp. The apparatus uses a lower wrapped shell that wraps around an upper wrapped shell, including two lines of comb teeth, a laser module, a switch device, and a coupling assembly. A laser splitter and a reflector are used to guide the laser light to the teeth of the comb, and the laser light is emitted along the groove of the teeth of the comb.

The hand-held apparatus of the prior art adopts the laser light to stimulate hair to grow, and destroys and excites the skin tissue to grow again to achieve the desired medical effects. However, there may exist the problem of excessive heat. When the user operates the apparatus improperly, the laser light may burn the skin. Furthermore, due to the unbalanced density of the teeth of the comb, the skin is exposed to the laser light unevenly.

Photomodulation apparatuses utilizing a light wavelength range having a medical effect of the prior art are only applied via large-sized medical apparatuses or with other expensive equipment. As such, it cannot be applied to general beauty treatments or health care for individual daily use. Because the photomodulation apparatuses of the prior art do not specify the treatment area, the photon dose cannot be easily controlled. Another related invention, applied for by the inventor of this invention, adopts a light source packaged by a chip on board to develop a hand-held photon dose modulation apparatus. The apparatus uses a setting controller to set the light source, the power rate of the photon, the shining period, the treatment area, the light source control, and the shining mode to achieve the goal of a hand-held photon dose modulation apparatus. The hand-held photon shining apparatus of the present invention utilizes related technology to control the photon dose. Therefore, the complexities of controlling the photon dose not exist.

### SUMMARY OF THE INVENTION

One particular aspect of the present invention is to provide a hand-held photon shining apparatus. The apparatus matches the curves of a human body and is ergonomically designed. The apparatus closely contacts the part of the human body being operated upon. The apparatus can be held using the user's hand thus avoiding the prior art's problem, the user's muscles tiring quickly.

The hand-held photon shining apparatus of the present invention utilizes a concave slot and a ring of flange formed at the opening of the concave slot to make the user aware of the shining location. Thereby, the problem and consequences of shifting the apparatus is avoided.

Furthermore, the hand-held photon shining apparatus of the present invention utilizes a chip on board to package the high power light-emitting element to reduce the width and the height of the apparatus. Therefore, the apparatus is easily held in the user's hand, which is especially useful for women or those men with small sized palms.

Furthermore, a built-in battery or an external power source can supply power to the hand-held photon shining apparatus of the present invention allowing the user to select either as a power source. When the external power source is adopted, the power line is connected below the photon shining apparatus to prevent the operation from being affected.

The hand-held photon shining apparatus of the present invention includes an assembly part having a shining opening (having a variety of bores) to be used upon the treatment area of the human body having different dimensions.

The hand-held photon shining apparatus includes a main body having a top surface, a bottom surface, and two side surfaces. A bottom concave part that corresponds to the curves of the human body is formed on the bottom surface. A battery receiving space located at the rear half part of the top surface of the main body is used for receiving at least one battery. A switch is located at the front half part of the top surface of the main body. A concave slot is formed on the bottom surface of the main body and is located within the bottom concave part. At least one light-emitting element is located in the slot bottom of the concave slot. By holding the main body by hand and covering the slot opening of the concave slot on the treatment area of the human body, and pressing the switch, the light-emitting element is controlled to shine upon the treatment area.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is a schematic view of the light source of the low energy photon therapy apparatus of the prior art;

FIG. 2A is a schematic view of the medical apparatus of the first embodiment of the prior art;

FIG. 2B is a schematic view of the medical apparatus of the second embodiment of the prior art;

FIG. 3 is a perspective view of the photon shining apparatus and its power converter of the present invention;

FIG. 3A is a side view of the photon shining apparatus without the power converter of the present invention;

FIG. 4 is a top view of the photon shining apparatus without the power converter of the present invention;

FIG. 5 is a bottom view of the photon shining apparatus of the present invention based on FIG. 4;

FIG. 6 is a side view of the photon shining apparatus of the present invention based on FIG. 4;

FIG. 7 is a side view displaying the linking relationship between the photon shining apparatus without the power converter of the present invention and an assembly part; and

FIG. 8 is a side view of the photon shining apparatus without the power converter of another embodiment of the present invention.

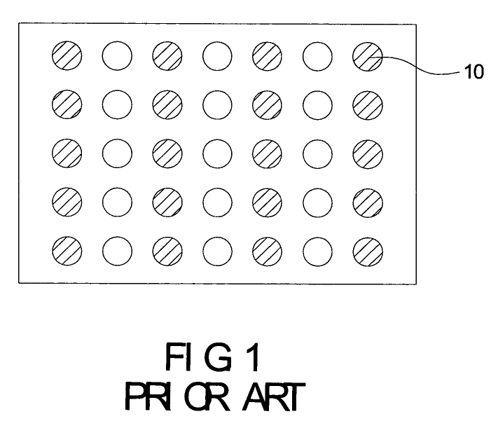

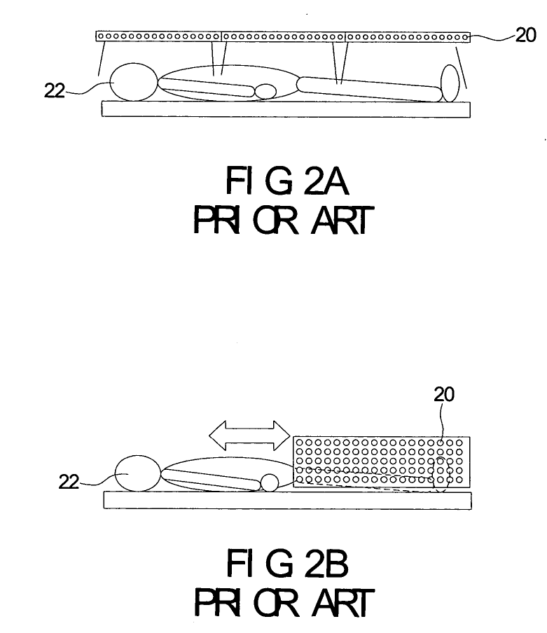

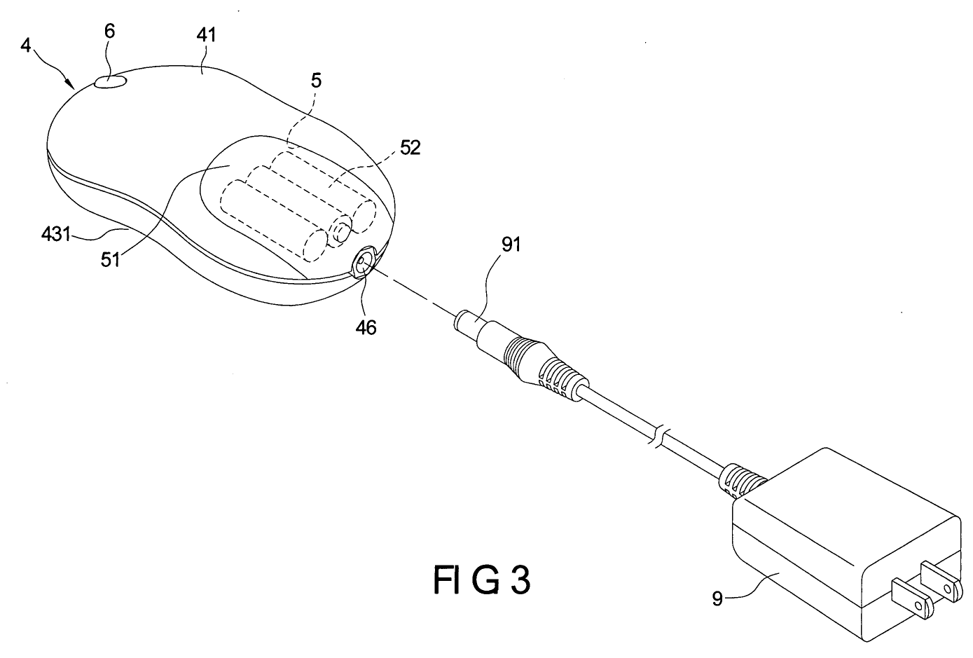

### DETAILED DESCRIPTION OF THE PREFERRED EMBODIMENTS

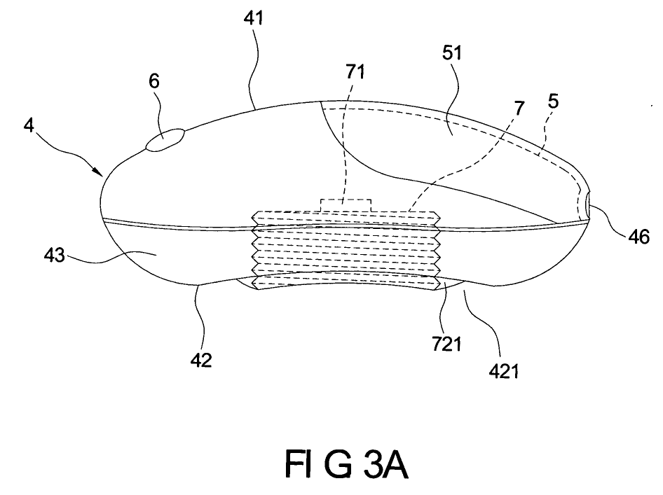

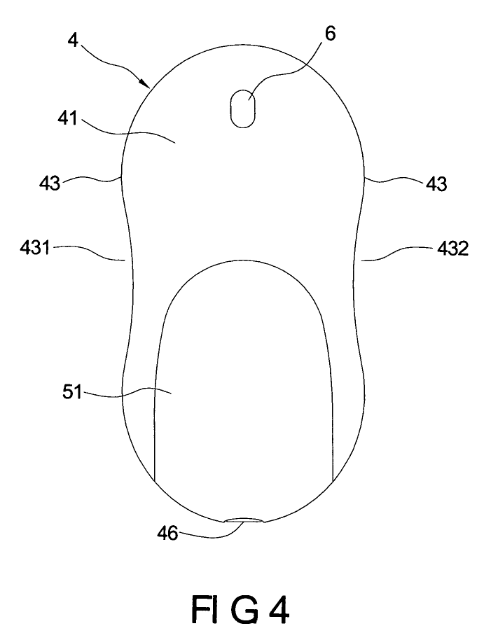

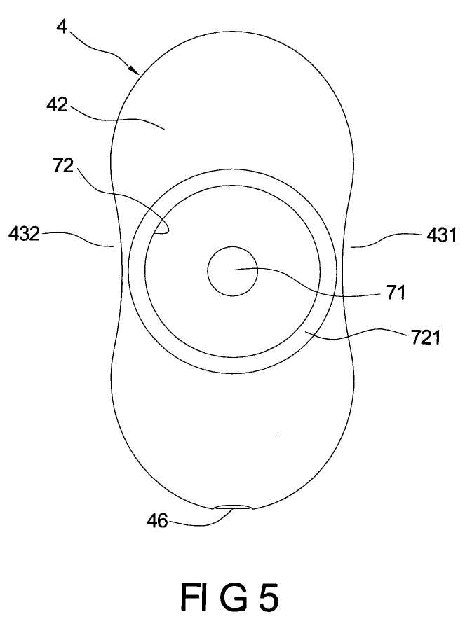

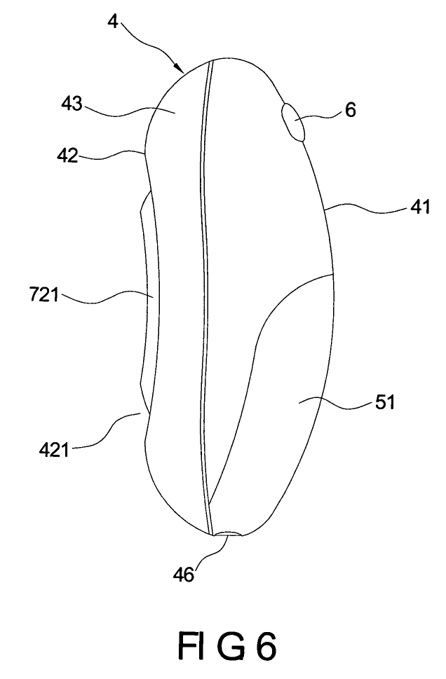

Please refer to FIGS. 3-7, which show a schematic view of the hand-held photon shining apparatus. The hand-held photon shining apparatus includes a main body 4, a battery receiving space 5, a switch 6, a concave slot 7, and at least one light-emitting element 71. For a preferred embodiment, an assembly part 8 and a power converter 9 having a power plug 91 are included.

The main body 4 has a top surface 41, a bottom surface 42, and a side surface having two sides 43. A bottom concave part 421 that corresponds to the curves of a human body is formed on the bottom surface 42 for contacting the human body closely. For a preferred embodiment, a first concave part 431 is formed on one of the two sides 43 of the main body 4. The first concave part 431 matches the curves of a human palm. For holding by the user's right hand, the right thumb buckles into the first concave part 431. Furthermore, a second concave part 432 is formed on another one of the two sides 43 of the main body 4. The second concave part 432 also matches the curves of a human palm so that the device may be held in the user's right hand, all the user's fingers, except the forefinger, buckle into the second concave part 432.

The battery receiving space 5 is located at the rear half part of the top surface 41 of the main body 4 and is used for receiving at least one battery 52. A battery cover 51 is covered above the battery receiving space 5. The battery 52 can be a general battery or rechargeable battery 42, such as a lithium battery, a Nickel-Cadmium battery, or a Nickel-Metal Hydride battery.

The switch 6 is located at the front half part of the top surface 41 of the main body 4. When the user's hand holds the main body 4, it is convenient for the forefinger to press.

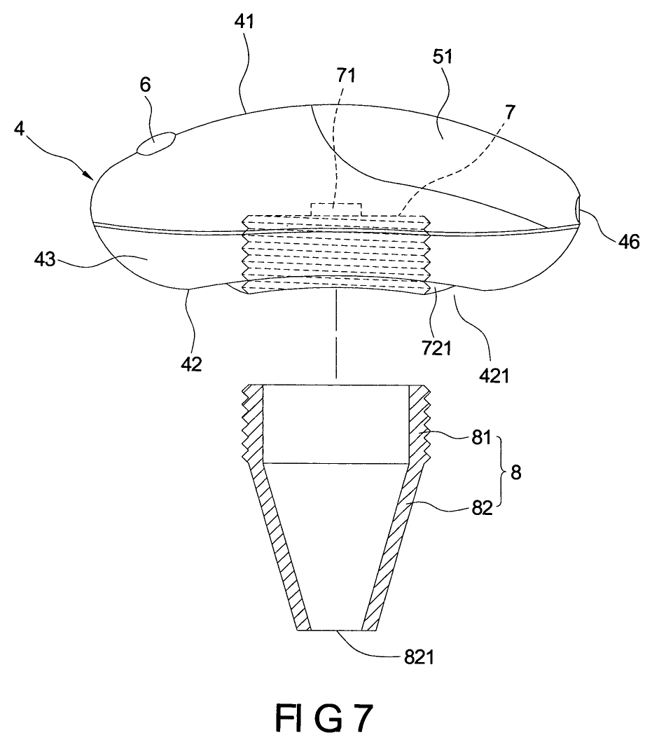

Please also refer to FIG. 7. The concave slot 7 is formed on the bottom surface 42 of the main body 4 and is located within the bottom concave part 421. The slot opening 72 of the concave slot 7 further includes a flange 721. The flange is formed and integrated into one piece according to the outline of the slot opening 72 of the concave slot 7, and is convenient for the user to sense the target location.

Still referring to FIG. 7, the light-emitting element 70 is located in the slot bottom of the concave slot 7 and is electrically connected with the battery 52 and the switch 6 via a control circuit (not shown in the figure) located in the main body 4. When the switch 6 is pressed, the light-emitting element 71 is controlled to shine upon the treatment area via a permanent-shining method, a glittering-shining method, or automatically stops shining after shining for a specified period.

The hand-held photon shining apparatus utilizes the chip on board to package the high power light-emitting element 71 to reduce the width and the height of the apparatus. Therefore, the apparatus is easily held in the user's hand, and is especially comfortable for those with small palms.

For a preferred embodiment, a power converter 9 used for linking with the household power outlet and an assembly part 8 used for reducing the size of the opening of the shining part are included.

The power converter 9 is used for linking with the household power outlet or another power supply. The power converter 9 can be a power line or a regulator, and a power plug 91 is located on one end of the power converter 9. A power plughole 46 is located at the lower part of one of the sides of the main body 4. The power plug 91 is electrically plugged into the power plughole 46 of the main body 4 to supply power. Because the power plughole 46 is located at the lower part of one of the sides of main body 4, it will not affect the operation of the apparatus.

Still referring to FIG. 7, the hand-held photon shining apparatus of another embodiment of the present invention further includes an assembly part 8 used for reducing or adjusting the size of the opening of the shining part. The assembly part 8 includes a column with a large diameter and a column with a small diameter (not shown in the figure) and link with each other. Part or the entire column with a large diameter has an external thread, and the inner part of the concave slot 7 has an inner thread that corresponds to the external thread for attaching the column with a large diameter and the column with a small diameter together in a screwing manner. As shown in FIG. 7, the assembly part 8 can also include a hollow assembly element 81 and a hollow deforming part 82 having a shining opening 821. The hollow assembly element 81 is assembled in the concave slot 7 of the main body 4 to make the light of the light-emitting element 71 shine from the shining opening 821. The hollow assembly element 81 has an external thread, and in the inner part of the concave slot 7 there is a corresponding inner thread for assembling and dissembling the assembly part 8. The deforming part 82 can be cone-shaped or angle cone-shaped. The free end of the deforming part 82 having a cone-shape or an angle cone-shape is the shining opening 821 that makes shining on a specific or small part of the human body more convenient.

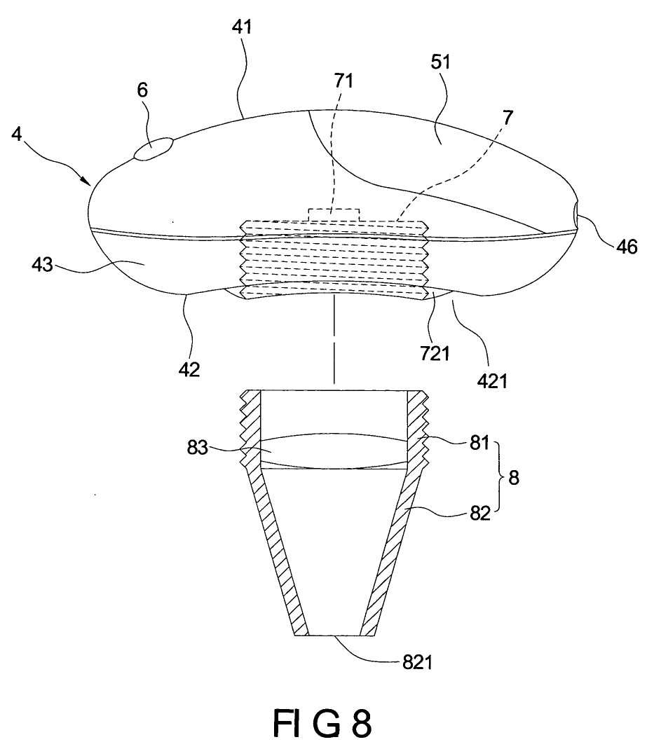

Please refer to FIG. 8, which shows another embodiment of the photon shining apparatus of the present invention. A light-gathering element 83 (or a light-gathering module, not shown in the figure) is installed in the assembly part 8 in FIG. 7. Because the power of the light-emitting element 71 is fixed and the assembly part 8 is installed, the distance between the light-emitting element 71 and the treatment area of the human body becomes larger and the illumination on the treatment area becomes smaller. In order to maintain the illumination, the light-gathering element 83 is installed in the assembly part 8. The light-gathering element 83 can be a light-gathering lens. After the light is gathered, the illumination shined on the treatment area of the human body is similar to the illumination before the assembly part 8 is installed. For a preferred embodiment, the light-gathering element is installed in the assembly element 81 of the assembly part 8.

The present invention has the following characteristics:

1. The bottom concave part 421 makes the hand-held photon shining apparatus contact the human body closely to achieve the shining effect.

2. Through the design of the first concave part 431, the second concave part 432, and the switch 6, the right thumb buckles into the first concave part 431, the forefinger presses the switch 6, and the other three fingers buckle into the second concave part 432. Therefore, the device has an ergonomic design and can be held conveniently in the user's right hand.

3. Through the design of the concave slot 7, the distance between the light-emitting element 7 and the treatment area of the human body can be maintained at a pre-determined distance (the depth of the concave slot). The hand-held photon shining apparatus contacts directly with the treatment area of the human body to assure the shining location.

4. Through the design of the slot opening 72 of the concave slot having a flange 721, it is convenient for the user to be aware of the shined location. Therefore, the user can prevent the problem of being unaware that the device has been moved accidentally.

5. Through the design of the chip on board of packaging the high power light-emitting element, the width and the height of the main body 4 are reduced. Therefore, the device can be held in the user's hand and is therefore convenient for user with small palms.

6. Through the design of the power plughole 46 and its related design, the hand-held photon shining apparatus can receive power from a built-in battery or an external power source. The power plughole 46 is located at the lower side of the main body 4 to prevent the operation of the device from being affected negatively.

7. Through the design of the assembly part 8, it is convenient to shine light on the treatment area of a specific or small part of the human body. According to the dimensions of the treatment area, the assembly part 8 having a shining opening 821 with a corresponding size is chosen to enhance the shining effect.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. A hand-held photon shining apparatus, comprising:
a main body having a top surface, a bottom surface, and two side surfaces, wherein a bottom concave part that corresponds to the curves of a human body is formed on the bottom surface;
a battery receiving space located at the rear half part of the top surface of the main body for receiving at least one battery;
a switch located at the front half part of the top surface of the main body;
a concave slot formed on the bottom surface of the main body and located within the bottom concave part; and
at least one light-emitting element located in the slot at the bottom of the concave slot;
wherein by holding the main body by hand and covering the slot opening of the concave slot over the treatment area of human body, the light-emitting element is controlled to shine upon the treatment area, when the switch is pressed.

2. The hand-held photon shining apparatus as claimed in claim 1, wherein a first concave part is formed at one side of the main body and the first concave part corresponds to the curves of a human palm.

3. The hand-held photon shining apparatus as claimed in claim 2, wherein a second concave part is formed at another side of the main body and the second concave part corresponds to the curves of a human palm.

4. The hand-held photon shining apparatus as claimed in claim 1, wherein the light-emitting element is packaged by chip on board.

5. The hand-held photon shining apparatus as claimed in claim 1, wherein the slot opening of the concave slot further comprises a flange.

6. The hand-held photon shining apparatus as claimed in claim 5, wherein the flange is formed and integrated into one piece according to the outline of the slot opening of the concave slot.

7. The hand-held photon shining apparatus as claimed in claim 1, wherein a power plughole is located at the lower part of one of the sides of the main body for attaching a power plug connecting with a household power outlet.

8. The hand-held photon shining apparatus as claimed in claim 1, further comprising an assembly part, wherein the assembly part comprises a column with a large diameter and a column with a small diameter that link with each other, and part or the entire column with a large diameter has an external thread and the inner of the concave slot has an inner thread that corresponds to the external thread for attaching the column with a large diameter and the column with a small diameter in a screwing manner.

9. The hand-held photon shining apparatus as claimed in claim 1, further comprising an assembly part, wherein the assembly part comprises a hollow assembly element and a hollow deforming part having a shining opening, and the assembly element is assembled in the concave slot of the main body.

10. The hand-held photon shining apparatus as claimed in claim 9, wherein the assembly element of the assembly part has an external spread, and the inner part of the concave slot has a corresponding inner thread for attaching to the external spread of the assembly element in a screwing manner.

11. The hand-held photon shining apparatus as claimed in claim 9, wherein the deforming part of the assembly part is cone-shaped or angle cone-shaped.

12. The hand-held photon shining apparatus as claimed in claim 9, wherein the assembly part further comprises a light-gathering element.

13. The hand-held photon shining apparatus as claimed in claim 12, wherein the light-gathering element is installed in the assembly element of the assembly part.
